(19) United States
(12) Patent Application Publication
(10) Pub. No.: US 2026/0337032 A1
(43) Pub. Date: Oct. 29, 2026

(54) PROCESS FOR PHOSGENE PRODUCTION WITH REUSE OF CARBON DIOXIDE FROM RECYCLING OF USEFUL MATERIAL

(71) Applicant: Covestro Deutschland AG, Leverkusen (DE)

(72) Inventors: Andreas Bulan, Langenfeld (DE); Sebastian Pohl, Grünstadt (DE); Gaston Zabalza, Montevideo (UY)

(21) Appl. No.: 19/110,289

(22) PCT Filed: Sep. 27, 2023
(86) PCT No.: PCT/EP2023/076631

§ 371 Date: Mar. 10, 2025

(30) Foreign Application Priority Data
Sep. 30, 2022 (EP) 22199231.6

(51) Int. Cl. C01B 32/80; C01B 32/40; C01B 32/50; C07C 269/06; C07C 271/06; C25B 1/04; C25B 11/032

(52) U.S. Cl. CPC C01B 32/80; C01B 32/40; C01B 32/50; C07C 269/06; C07C 271/06; C25B 1/04; C25B 11/032

(57) ABSTRACT

A process for producing phosgene, especially for the reaction of phosgene with an organic amino compound to give organic isocyanate is provided. In this process, at least one carbon dioxide gas stream that has been formed from a hydrolysis of organically modified carbamate and may have been freed of secondary constituents is reductively converted to carbon monoxide. The resulting carbon monoxide is reacted with chlorine in a phosgene synthesis to give phosgene. The organically modified carbamate for the process can preferably be obtained from a chemolysis of end-of-life polyurethane material, especially with alcohols, amines or amino alcohols as chemolysis reagent.

### CROSS REFERENCE TO RELATED APPLICATIONS

This application is a U.S. national stage application, filed under 35 U.S.C. § 371, of International Application No. PCT/EP2023/076631, which was filed on Sep. 27, 2023, and which claims priority to European Patent Application No. 22199231.6, which was filed on Sep. 30, 2022. The entire contents of each are hereby incorporated by reference into this specification.

### FIELD

The invention relates to a process for producing phosgene, especially for the reaction of phosgene with organic amino compound to give organic isocyanate, in which at least one carbon dioxide gas stream that has been formed from a hydrolysis of organic carbamate and optionally freed of secondary constituents is converted to carbon monoxide, and the resulting carbon monoxide is converted to phosgene with chlorine in a phosgene synthesis.

### BACKGROUND

The recycling of carbon dioxide (also referred to hereinafter as CO₂) into the value chain is a contribution to sustainability. In the past, there were some efforts to make CO₂ utilizable, for example, for the production of carbon monoxide (the latter also being referred to hereinafter as CO).

Document EP 3 744 812 B1 states that CO₂ formed in the pyrolysis of useful polyurethane material and CO₂ formed in the combustion of useful polyurethane material is converted to CO with reduction of CO₂ in a CO₂ electrolysis. The CO obtained is reacted with chlorine to give phosgene, which serves as a starting material for polyurethane in the preparation of organic isocyanates. In particular, the CO₂ obtained in combustion has to be subjected to a complex cleaning method before it can be used in an electrochemical CO₂ reduction.

In the process of recycling CO₂ into the production of isocyanate compounds according to document WO 2021/089737 A1, CO₂ from an external source, for example a combustion of useful polyurethane material, is converted to CO with hydrogen in a reverse water-gas shift reaction (RWGS reaction). Here too, the CO₂ has to be cleaned in a complex manner prior to use in the RWGS reaction.

A further option for recycling of CO₂ into the production of isocyanate compounds is offered by the process according to document WO 2022/167387 A1. Herein, the CO₂ formed in a steam reforming process and additional CO₂ from a further CO₂ source are recycled back into the steam reforming process. A further CO₂ source mentioned is, for example, the CO₂ obtained in cement production or in incineration of wastes. CO₂ from these sources must likewise be cleaned in a complex manner prior to use in the steam reforming process.

### SUMMARY

It is an object of the present invention to reduce the process complexity of the prior art CO₂ utilization processes in order to make the processes more economic and/or more environmentally benign. Overall, a sustainable process for production and reuse of polyurethane material is to be provided.

The “sustainability” of a process is understood by those skilled in the art according to the definition of sustainability (sustainable development) coined by the UN in the Brundtland Report of the “World Commission on Environment and Development”, this being that the execution of the process in the present makes the smallest possible contribution, or none at all, to compromising the ability of future generations to meet their own needs, in particular needs in respect of the use of resources such as fossil raw materials and especially in respect of the conservation of living space, for example the protection of the earth's atmosphere.

It has been found that the CO₂ formed in the hydrolysis of organically modified carbamate compounds (especially the CO₂ formed in the hydrolysis of an organically modified carbamate obtained from a chemolysis of a polyurethane material) has very high purity and can be provided with a low level of use of economic and/or environmental resources for the reductive conversion to CO.

The invention thus firstly provides a process for producing phosgene (especially for the preparation of organic isocyanate) by at least the following steps:
producing a CO₂ gas stream by at least the following steps:
   providing at least one organically modified carbamate;
   hydrolyzing the at least one organically modified carbamate provided to form at least organic amino compound and CO₂;
   separating off the CO₂ formed to obtain at least one CO₂ gas stream;
preferably cleaning the CO₂ gas stream to remove secondary constituents (especially secondary constituents selected from at least one compound from the list formed by alcohols, polyethers, hydrocarbons, organic amines, water, sulfur compounds, dust, oxygen and nitrogen) by at least one cleaning method selected from condensation, adsorption, catalytic gas cleaning or gas scrubbing, to obtain a cleaned carbon dioxide;
reducing CO₂ in the at least one optionally cleaned CO₂ gas stream produced to carbon monoxide;
synthesizing phosgene at least from the carbon monoxide obtained by said reduction and chlorine.

Documents DE 197 19 084 A1, EP 0 031 538 A2, WO 2022/171586 A1 and WO 2022/128871 A1, for example, disclose that chemolysis of useful polyurethane material can give organic amines and polyols as such chemical compounds from the useful polyurethane material that are reutilizable for the production of polyurethane material.

One approach to physical recycling of polyurethane material in this context is, for example, an alcoholysis or glycolysis in which the urethane group is reacted with alcohol or glycol by transesterification or transurethanization, respectively:

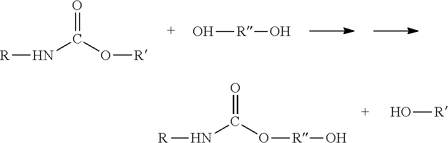

In addition, the urethane group can be reacted with an amine to form a ureylene group:

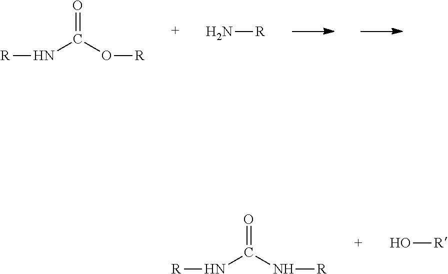

There is no description anywhere of the particular suitability of CO₂-containing offgas from the hydrolysis of organically modified carbamates, or from the process of chemolysis of polyurethane material, for reutilization.

“Polyurethane material” in the context of the present invention is the polyaddition products (occasionally also referred to, albeit not entirely correctly, as polycondensation products) that are obtained by reaction of polyfunctional isocyanates (=isocyanate component in the polyurethane preparation) and polyols (=polyol component in the polyurethane preparation). Polyurethane material generally contains not only the polyurethane basic structure outlined above but also other structures, for example structures having urea bonds. The presence of such structures at variance from the pure polyurethane base structure in addition to polyurethane structures does not depart from the scope of the present invention. Polyurethane material is in particular polyurethane foams obtained by reaction of polyfunctional isocyanates with polyols in the presence of a blowing agent.

### BRIEF DESCRIPTION OF THE DRAWING

FIG. 1A schematic overview of the overall process for producing low-emission toluene diisocyanate (TDI), CO production by RWGS, which is heated with bio-natural gas, HCl recycling by Cl₂ production via thermocatalytic gas phase oxidation (Deacon) of HCl and H₂ provision from a water electrolysis and CO₂ provision from chemolysis of polyurethane material (TDI-based foams) with diethylene glycol (PEG) as chemolysis reagent and hydrolysis of the resulting organically modified carbamates in a separate step.

FIG. 2A schematic overview of the overall process for reducing a low-emission toluene diisocyanate (TDI), comprising rWGS reaction, chlorine production, PU production, use, and utilization of the polyurethane material waste and recycling via hydroglycolysis and use of the CO₂ for the RWGS reaction.

### DETAILED DESCRIPTION

In the terminology of the present invention, the term isocyanates encompasses all isocyanates known to a person skilled in the art in connection with polyurethane chemistry, such as, in particular, tolylene diisocyanate (TDI; produced from tolylenediamine, TDA), the di- and polyisocyanates of the diphenylmethane series (MDI; produced from the di- and polyamines of the diphenylmethane series, MDA), pentane 1,5-diisocyanate (PDI; produced from pentane-1,5-diamine, PDA), hexamethylene 1,6-diisocyanate (HDI; produced from hexamethylene-1,6-diamine, HDA), isophorone diisocyanate (IPDI; produced from isophoronediamine, IPDA) and xylylene diisocyanate (XDI; produced from xylylenediamine, XDA). The expression “an isocyanate” does of course also encompass embodiments in which two or more different isocyanates (e.g. mixtures of MDI and TDI) were used in the production of the polyurethane foam, unless explicitly stated otherwise, for instance by the wording “exactly one isocyanate”. The entirety of all isocyanates used in the production of the polyurethane foam is referred to as the isocyanate component (of the polyurethane foam). The isocyanate component comprises at least one isocyanate. Analogously, the entirety of all polyols used in the production of the polyurethane foam is referred to as the polyol component (of the polyurethane foam). The polyol component comprises at least one polyol.

In the terminology of the present invention, the term polyols encompasses all polyols known to a person skilled in the art in connection with polyurethane chemistry, such as, in particular, polyether polyols, polyester polyols, polyetherester polyols and polyethercarbonate polyols. The expression “a polyol” does of course also encompass embodiments in which two or more different polyols were used in the production of the polyurethane foam. Therefore, if reference is made hereinafter, for example, to “a polyether polyol” (or “a polyester polyol” etc.), this terminology does of course also encompass embodiments in which two or more different polyether polyols (or two or more different polyester polyols etc.) were used in the production of the polyurethane material.

“Organically modified carbamate” in the terminology of the present invention refers to organic urethane compounds. These include both polyurethane material and preferably those organically modified carbamates that are formed in the chemolysis of polyurethane material by reaction with a chemolysis reagent.

An “organic compound” contains at least one covalent carbon-hydrogen bond in the molecule. An organic amino compound is accordingly an organic substance containing, as a chemical compound, at least one amino group and additionally at least one covalent carbon-hydrogen bond in the molecule. An organically modified carbamate is defined mutatis mutandis.

According to the invention, a “chemolysis” means the chemical conversion of polyurethane material with cleavage of the polyurethane polymer structure by transesterification (also referred to as transurethanization) or by formation of a ureylene group (also: carbonyldiimino group, *—NH—C(═O)—NH—*).

“Chemolysis reagent” means a reagent that enters into a chemical reaction for performance of a chemolysis with the urethane bond as reactive group of the polyurethane material.

In the context of a preferred embodiment of the process, the organically modified carbamate is provided by a chemolysis of polyurethane material. For the provision of the organically modified carbamate, it is sufficient in the context of this embodiment when the organically modified carbamate is a direct process product of a chemolysis of polyurethane material. This means that it is sufficient in the case of for the execution of the step of providing the organically modified carbamate merely to take said organically modified carbamate from a storage vessel or from a feed conduit in the context of a delivery as raw material. In this case, the phosgene producer, as executor of the process of the invention, does not itself execute the chemolysis of the polyurethane material for production of the organically modified carbamate, but merely ensures that the organically modified carbamate provided has been produced accordingly by chemolysis by a supplier and is the process product thereof. A preferred embodiment of the process is thus characterized in that the organically modified carbamate provided is at least one direct process product of a chemolysis of polyurethane material, comprising at least the following process steps:
providing polyurethane material;
reacting the polyurethane material provided with at least one chemolysis reagent (especially selected from (a) a primary or secondary organic amine, (b) an amino alcohol having a primary or secondary amino group or (c) an alcohol having at least one hydroxyl group) to form at least one organically modified carbamate.

The polyurethane material provided may in principle be any kind of polyurethane product, i.e. either polyurethane foams or polyurethane products from what are called CASE applications. The polyurethane foams may be either flexible foams or rigid foams, preference being given to flexible foams (for example from used mattresses, furniture cushioning or car seats). In the case of polyurethane products from CASE applications, preference is given to polyurethane elastomers, polyurethane adhesives and polyurethane coatings. Of all polyurethane products, flexible polyurethane foams are particularly preferred. The polyurethane material provided is provided, for example, from polyurethane material waste of corresponding end-of-life polyurethane products (for example mattresses, cushions or insulation materials).

In addition, preference is given to polyurethane material based, with regard to the isocyanate component, on an isocyanate selected from the group consisting of tolylene diisocyanate (TDI), the di- and polyisocyanates of the diphenylmethane series (MDI), pentane 1,5-diisocyanate (PDI), hexamethylene 1,6-diisocyanate (HDI), isophorone diisocyanate (IPDI) and xylylene diisocyanate (XDI), and mixtures of two or more of the aforementioned isocyanates. Particular preference is given to polyurethane material based, with regard to the isocyanate component, on TDI or a mixture of TDI and MDI. Very particular preference is given to polyurethane material based, with regard to the isocyanate component, solely on TDI.

In addition, preference is given to polyurethane material based, with regard to the polyol component, on a polyol selected from the group consisting of a polyether polyol, a polyester polyol, a polyetherester polyol, a polyethercarbonate polyol and a mixture of two or more of the aforementioned polyols.

The provision of the polyurethane material preferably comprises preparatory steps for the subsequent reaction with the chemolysis reagent. These especially involve mechanical comminution of the polyurethane material. Such preparatory steps are known to the person skilled in the art; reference is made, for example, to the review article by Simón, Borreguero, Lucas and Rodríguez in Waste Management 2018, 76, 147-171.

It has been found to be particularly preferable when the polyurethane material provided has been subjected to inertization, at least comprising removal of gaseous oxygen surrounding the polyurethane material and/or contained in the pores thereof. The performance of inertization is advisable in particular when the polyurethane material is in the form of a powder, granules or a foam. Inertization of the provided polyurethane material prior to reaction with the chemolysis reagent achieves particularly high purity of the CO₂ gas stream obtained in the hydrolysis. If, for example, there should be no inertization in the case of a polyurethane material in the form of a foam, the CO₂ obtained in the hydrolysis will have an elevated concentration of N₂ and especially of O₂ that has to be removed from the CO₂ gas stream with a high degree of complexity.

In a preferred embodiment, the inertization additionally encompasses a step of feeding at least one inert gas into the polyurethane material (especially nitrogen, carbon dioxide, argon or helium, most preferably carbon dioxide).

Inertization of the polyurethane material provided is more preferably conducted by at least the following steps:
degassing the polyurethane material, with application of a first pressure of not more than 960 mbar_{(abs.)} and a temperature of not more than 120 °C in a vessel, and removal of the gas in the headspace gas phase via a gas removal device. The gas is removed by applying as a first pressure in the vessel a reduced pressure of less than 700 mbar_{(abs.)}, preferably a pressure of 0.1 mbar_{(abs.)} to 100 mbar_{(abs.)}, and removing the gas via the gas removal device.

After the gas has been removed, at least one inert gas (especially nitrogen, carbon dioxide, argon or helium, most preferably carbon dioxide) is fed in to establish a pressure of preferably not more than 1.8 bar_{(abs.)}, more preferably of atmospheric pressure.

Performance of the degassing and of the overall inertization is described in document WO 2022/128871 A1, to which reference is made here explicitly and in full.

It is likewise possible in the context of a further embodiment of the invention when, in order to provide the organically modified carbamate, the aforementioned chemolysis conversion is performed as an integral step of the process for producing phosgene by the phosgene producer itself, and then the resultant organically modified carbamate is fed onward to the hydrolysis to form CO₂. Therefore, a corresponding preferred embodiment of the process is characterized in that said CO₂ gas stream (31) is produced by chemolysis of polyurethane material, comprising at least the steps of
providing polyurethane material;
reacting the polyurethane material provided with at least one chemolysis reagent (especially selected from (a) a primary or secondary organic amine, (b) an amino alcohol having a primary or secondary amino group or (c) an alcohol having at least one hydroxyl group) to form at least one organically modified carbamate;
hydrolyzing the organically modified carbamate formed beforehand to form at least organic amino compound and CO₂;
separating off the CO₂ formed to obtain at least one CO₂ gas stream.

In the context of the aforementioned embodiment of the invention, the hydrolysis step can be effected as a separate process step from the conversion of the polyurethane material and/or simultaneously in one step. In a simultaneous sequence, a mixture comprising chemolysis reagent and additionally sufficient water is used.

It is generally preferable when the organically modified carbamate provided is formed by a reaction of the polyurethane material provided with at least one chemolysis reagent with exclusion of oxygen or is a direct process product of such a reaction. This means that the reaction is performed in an inert gas atmosphere (especially in a nitrogen, carbon dioxide, argon or helium atmosphere, more preferably a carbon dioxide atmosphere). It is also preferable to free the chemolysis reagents used (water and chemolysis reagent) of oxygen by inert gas saturation. In this connection, it is very particularly preferable when the polyurethane material provided is inertized as described above.

The provision of organically modified carbamate, in a preferred embodiment, is achieved by at least the following process steps:
providing polyurethane material;
reacting the polyurethane material provided with at least one chemolysis reagent (especially selected from (a) a primary or secondary organic amine, (b) an amino alcohol having a primary or secondary amino group or (c) an alcohol having at least one hydroxyl group) to form a composition comprising at least one organically modified carbamate;
blending the composition obtained beforehand, without prior removal of any water present in that composition, with an organic solvent which is incompletely miscible with the chemolysis reagent used beforehand, and phase separation into a carbamate phase (containing organically modified carbamate) and a solvent phase.

In this embodiment, the resultant carbamate phase is introduced into the hydrolysis step.

The aforementioned organic solvent to be used should be incompletely miscible with the chemolysis reagent used in the chemolysis. This means that there must be a miscibility gap under the conditions that exist for the above blending such that phase separation is possible. The organic solvent is more preferably selected from the group consisting of aliphatic hydrocarbons, alicyclic hydrocarbons, aromatic hydrocarbons, and mixtures of two or more of the aforesaid organic solvents.

In the integration of the chemolysis as a process step in a further preferred embodiment of the phosgene production of the invention, it is particularly preferable when said reaction of polyurethane material to give said organically modified carbamate and said hydrolysis of the organically modified carbamate are implemented together in one process step.

In general, water for the hydrolysis and chemolysis reagent for the chemolytic reaction in the process of the invention are preferably used in superstoichiometric amounts, no matter whether the chemolytic reaction takes place alone or in one step in combination with hydrolysis. This means that water for the hydrolysis is used in an amount theoretically sufficient to hydrolyze all the polyurethane bonds in the polyurethane material provided with release at least of carbon dioxide and of organic amino compound. Similarly, the use of superstoichiometric amounts of chemolysis reagent means that it is used in an amount theoretically sufficient to convert all the polyurethane bonds in the polyurethane material provided to form organically modified carbamates. In the case of use of the proportions by mass of water and chemolysis reagent preferred in accordance with the invention that are described hereinafter, both are regularly the case.

The chemolytic conversion is preferably conducted such that the mass ratio of chemolysis reagent (used in total) and water (used in total) on the one hand to the polyurethane material provided on the hand (i.e. [m(chemolysis reagent)+m(water)]/m(polyurethane product), m=mass) is in the range from 0.5 to 2.5, more preferably from 1.0 to 1.3, where the mass of the water is 2.0% to 10% of the mass of the chemolysis reagent. The quantitative figures in respect of water relate here to the water added as a reagent for the hydrolysis. By comparison, any amounts of water emanating from moisture that are present in any case in the chemolysis reagent used and/or in the polyurethane material used are low. Moisture in the chemolysis reagent used or in the polyurethane material used means traces of moisture as can occur on an industrial scale. The mass of water here is further preferably 4.0% to 10.0%, especially preferably 5.0% to 7.0%, of the mass of the chemolysis reagent used.

The chemolytic reaction and the hydrolysis can preferably be conducted as a chemolysis of the polyurethane material with a chemolysis reagent and water (preferably in the presence of a catalyst) at a temperature in the range from 130 °C to 220 °C, where the mass ratio of chemolysis reagent and water on the one hand to the polyurethane material on the other hand is in the range from 0.5 to 2.5 and the mass of water is 4.0% to 10% of the mass of the polyurethane material.

In the context of common implementation of chemolysis and hydrolysis in one process step, it has been found to be particularly preferable not to add the water used for the hydrolysis right at the start of the reaction time, or at least not to add it completely. It has been found to be useful at first to mix only a small proportion, namely 2% to 4%, if any, of the total amount of water to be used with the other reactants (i.e. at least chemolysis reagent and polyurethane material), and to add the remaining or total amount of water over the course of the further reaction time. The water for the hydrolysis of the organically modified carbamates formed as intermediates is added continuously or in portions at intervals in such a way that the boiling temperature of the reaction mixture always remains within the specified ranges, in particular within the particularly preferred range of 165 °C to 185 °C. The metering time of the water is preferably in the range from 1.0 hour to 5.0 hours (depending on the boiling point of the chemolysis reagent used). In a preferred embodiment, a mass ratio of chemolysis reagent (used in total) and water (used in total) on the one hand to polyurethane material on the other hand [m(chemolysis reagent+water)/m(polyurethane material)] in the range from 1.1 to 1.3 is used. If the water is added not all at once but gradually as described above, this applies to the amount of water used altogether.

The amount of water which is used in the context of common implementation of chemolysis and hydrolysis in one process step is preferably 5.0% to 7.0% of the mass of the chemolysis reagent used; this is especially true in conjunction with the aforementioned range from 1.0 to 1.3 for the mass ratio [m(chemolysis reagent+water)/m(polyurethane material)]. If the water is added not at once but gradually as described above, this applies to the total amount of water used in this step.

In the context of common implementation of chemolysis and hydrolysis in one process step, it is unnecessary to add all the water right at the start. In this case, the above-stated ranges for the amount of water based on the mass of the chemolysis reagent relate to the amount of water which is added in total by the end of the reaction time. The same applies if the chemolysis reagent is added gradually.

In particular, it is also possible
(I) first to admix the polyurethane material solely with chemolysis reagent or with the chemolysis reagent and a first portion of the water, and then
(II) to add water or a second portion of the water, especially only after the polyurethane material has gone into solution.

The expression “gone into solution” in this connection does not necessarily imply the existence of a “true” solution in the sense of a completely homogeneous mixture. It may quite possibly be the case that there is a cloudy “solution” of the polyurethane material; this does not leave the scope of the present invention.

In the course of performance of the common implementation of chemolysis and hydrolysis in the aforementioned steps (I) and (II), it is especially preferable, in step (II), to add the water or the second portion of the water continuously or in portions such that the temperature of the liquid phase during step (II) differs by a maximum of 20 °C, preferably by a maximum of 15 °C, more preferably by a maximum of 10 °C, even more preferably by a maximum of 5.0 °C and very exceptionally preferably by a maximum of 1.0 °C from the temperature of the liquid phase from step (I). What this achieves is that the temperature is always high enough to assure progression of the chemolytic reaction.

Performance of a chemolysis in combination with a hydrolysis in one step is described, for example, in document WO 2022/171586 A1 using the example of an alcohol having at least one hydroxyl group as chemolysis reagent. Reference is made here to this document explicitly and in full.

Performance of a chemolysis with a hydrolysis as a separate step is described, for example, in documents U.S. Pat. No. 4,336,406 and WO 2020/260387 A1, in each case using the example of an alcohol having at least one hydroxyl group as chemolysis reagent. Reference is made here to this document explicitly and in full.

Chemolysis can generally, optionally in combination with hydrolysis, be conducted in any reactor known for such a purpose in the specialist field. Especially suitable chemolysis reactors are stirred tanks (stirred reactors) and tubular reactors.

In a further embodiment, it is particularly preferable to assist chemolysis, as the reaction of polyurethane material with chemolysis reagent (for example alone or in one step in combination with hydrolysis), by the use of at least one catalyst. The catalyst used in this preferred embodiment is more preferably at least one compound selected from carbonate, hydrogencarbonate, orthophosphate, monohydrogenorthophosphate, metaphosphate, hydroxide, organic amine (especially diethanolamine), organometallic compounds (especially titanium tetrabutoxide, tin octanoate or dibutyltin dilaurate) or mixtures of two or more of the aforementioned catalysts. Very particular preference is given to using at least one metal salt selected from a carbonate, a hydrogencarbonate, an orthophosphate, a monohydrogenorthophosphate, a metaphosphate or a mixture of two or more of the aforementioned metal salts as catalyst.

Orthophosphates are the salts of orthophosphoric acid, H₃PO₄, where all protons have been eliminated (═PO₄³⁻). Monohydrogenorthophosphates are the salts of orthophosphoric acid where two protons have been eliminated (═HPO₄²⁻). Metaphosphates are condensation products of orthophosphoric acid having the empirical formula [(PO₃)⁻]ₙ where n denotes a natural number (especially 3 or 4).

If a catalyst is used in a chemolysis step, it is preferably used in such an amount that its mass is 0.1% to 3.5% of the mass of the polyurethane material provided.

If a catalyst is used in the form of an aqueous solution, the water used as solvent should likewise be taken into account in the quantitative figures given above for the use of water in the hydrolysis, i.e. the amount of water additionally to be used if required in the hydrolysis should be reduced correspondingly.

It is further preferable also to use at least one of the aforementioned catalysts for the hydrolysis.

In a further embodiment, it is particularly preferable to perform the chemolysis (for example alone or preferably in one step in combination with the hydrolysis) as a reaction of polyurethane material with at least one chemolysis reagent selected from (a) a primary or secondary organic amine, (b) an amino alcohol having a primary or secondary amino group or (c) an alcohol having at least one hydroxyl group.

A suitable alcohol having at least one hydroxyl group as chemolysis reagent is preferably at least one alcohol having at least two hydroxyl groups. In this context, it is particularly preferable in turn when the chemolysis reagent is selected from ethylene glycol, diethylene glycol, propylene glycol, dipropylene glycol, methyl glycol, triethylene glycol, glycerol, 2-methylpropane-1,3-diol or mixtures of two or more of the aforementioned alcohols.

A suitable amino alcohol having a primary or secondary amino group as chemolysis reagent is preferably at least one amino alcohol selected from ethanolamine, N-methylethanolamine, 3-amino-1-propanol.

A suitable primary or secondary organic amine as chemolysis reagent is preferably at least one aliphatic primary or aliphatic secondary organic amine. These primary or secondary amines are preferably mono- and/or diamines. Ethylene-1,2-diamine, 1,4-diaminobutane, hexamethylene-1,6-diamine or a mixture of two or more of these are chemolysis reagents that are particularly preferred as primary or secondary organic amine.

Said chemolytic reaction of polyurethane material with chemolysis reagent to give said organically modified carbamate is preferably conducted at a temperature within a range from 140 °C to 220 °C, preferably 170 °C to 200 °C. This temperature should likewise preferably also be observed in the case of simultaneous conversion of polyurethane material with hydrolysis in one step. There are no particular demands with regard to pressure. The reaction can be conducted either at reduced or elevated pressure; for example at a pressure of 200 mbar(abs.) to 2000 mbar(abs.), preferably 500 mbar(abs.) to 1500 mbar(abs.), more preferably 900 mbar(abs.) to 1300 mbar(abs.) and especially at ambient pressure. Pressure figures in the context of the present invention are always expressed as absolute pressures, identified by a subscript “abs.” that follows the unit of pressure (for example, an absolute pressure of 900 mbar is quoted as “900 mbar_{(abs.)}”).

In a further embodiment, preference is given to those processes in which the CO₂ gas stream obtained is at a temperature of 140 °C to 220 °C, preferably of 170 °C to 200 °C.

Cleaning of the carbon dioxide obtained to remove secondary constituents, especially nitrogen, oxygen, chemolysis reagent (for example diethylene glycol), sulfur compounds, dust and water, can preferably be effected by means of at least one cleaning method selected from condensation, adsorption, catalytic gas cleaning or gas scrubbing to obtain a cleaned carbon dioxide (31a). However, the CO₂ gas stream obtained by the process of the invention has an excellent degree of purity for further utilization in the reduction of CO₂. Optionally before the reduction of CO₂, just a few cleaning steps are required for workup of the CO₂. These can be conducted easily and effectively. The CO₂ obtained from the hydrolysis in the CO₂ gas stream contains, as predominant secondary constituents, chemolysis reagent, and also organic amino compound formed in the chemolysis and polyol. These constituents can effectively be removed by cleaning (4) of the CO₂ gas stream (31) in which at least the CO₂ gas stream is conducted through multiple condensers and cooled, where the organic and aqueous components present as a secondary constituent in the gas stream condense and are separated off in liquid form, and, on conclusion of the cleaning (4), a cleaned CO₂ gas stream (31a) is sent to the reduction (6) to form carbon monoxide.

A further improvement in cleaning of the CO₂ gas stream is achieved when a combination of condensation and gas scrubbing is conducted.

The carbon monoxide required for the synthesis of phosgene is prepared by reduction (6) of CO₂ in the at least one optionally cleaned CO₂ gas stream (31, 31a) produced as described above to give carbon monoxide (21a). In a further preferred embodiment of the process, the reduction of CO₂ is implemented at least by a process selected from
A) the electrochemical reduction of CO₂, preferably using electricity produced from renewable energy,
B) a reformer process in which methane (preferably biomethane) and steam are converted to carbon monoxide at a temperature of at least 500 °C with addition of at least CO₂, with supply of thermal energy,
C) conversion of hydrogen and CO₂ in a reverse water-gas shift RWGS reaction zone to carbon monoxide.

“Renewable energy” is understood by those skilled in the art to mean energy from an inexhaustible energy source, for example wind energy, hydro energy, bioenergy (e.g. conversion of biogas or biomass to power) or solar energy. Suitable renewable energy is therefore most preferably either wind power, solar energy, hydro power or mixtures thereof.

In the execution of a reduction of CO₂, it is preferable when the optionally cleaned CO₂ gas stream, for the reduction (6), is introduced into an electrolysis apparatus and reduced to carbon monoxide at an electrode, preferably at a gas diffusion electrode (more preferably using electricity produced from renewable energy).

The CO₂ electrolysis may be, for example, a high-temperature electrolysis which is operated at a temperature of more than 600 °C, possibly with the addition of water for production of synthesis gas. High-temperature electrolyses are known in principle and available on the market, e.g. from Haldor Topsoe, eCOs®. High-temperature electrolysis forms oxygen at the anode. A disadvantage of the known high-temperature electrolysis is its poor scale-up capability, so that for larger volumes of CO, for example more than 1 t/h CO, low-temperature electrolysis is currently still preferable.

If the CO₂ electrolysis is operated as a low-temperature electrolysis, the electrolysis takes place at a temperature below 150 °C.

With all CO₂ electrolyses, the CO₂ gas is fed to the cathode space.

In the case of low-temperature electrolysis, CO₂ is in particular converted to carbon monoxide and possibly hydrogen at a gas diffusion electrode. At the same time, O₂ or, in some cases, alternatively chlorine as well can be generated at the anode. If chlorine is generated at the anode, this chlorine can be fed to the phosgene synthesis and thus to the isocyanate production as a further feedstock. The person skilled in the art is aware, for example from document WO 2021/069470 A, of electrodes and a method of executing an electrochemical reduction of CO₂. More preferably, the electrochemical reduction of CO₂ is performed by a method of WO 2021/069470 A. Reference is made herein to this document explicitly and in full. In this preferred electrolytic process for production of carbon monoxide, carbon monoxide, optionally hydrogen and chlorine are obtained by electrochemical conversion of carbon dioxide and alkali metal chloride solution. This preferred electrolysis process is characterized in that the carbon dioxide is electrochemically reduced at a gas diffusion electrode as cathode in an aqueous alkali metal chloride-containing solution as catholyte and, at the same time, chlorine is produced anodically from an aqueous alkali metal chloride-containing solution as anolyte, where the alkali metal salt of carbonic acid formed in the catholyte, selected from alkali metal carbonate, alkali metal hydrogencarbonate or mixtures thereof, is then reacted with hydrogen chloride to give carbon dioxide and alkali metal chloride, and the carbon dioxide released is returned to the cathode space for the gas diffusion electrode and the alkali metal chloride produced is returned either to the anode space and/or to the cathode space.

According to the known principles, an MEA (membrane-electrode assembly) concept can also be used in low-temperature electrolysis. In this case, a catalyst is applied to the membrane. An upstream gas diffusion layer regulates gas and liquid transport. This can be effected on both the anode and the cathode side. It is also possible to bring a gas diffusion electrode into direct contact with the membrane.

The gas diffusion electrode used may be installed in the electrolysis cell in a zero-gap or else in a finite-gap arrangement. A preferred arrangement for a low-temperature electrolysis of CO₂ is described in document WO 2020/057998 A1, to which reference is made explicitly and in full.

The cathode space, or the gas diffusion electrode stored therein, can be supplied with an excess of CO₂. “Excess” means introducing more CO₂ than would be necessary for the stoichiometric conversion on the basis of the flowing electric current. A gas mixture consisting of unreacted CO₂, CO and H₂ thus leaves the cathode space.

A suitable reformer process in which methane (preferably biomethane) and steam are converted to carbon monoxide at a temperature of at least 500 °C with addition of at least said CO₂ and with supply of thermal energy is described in the PCT application with application number PCT/EP2022/052267, to which reference is made explicitly and in full. This process relates to the preparation of carbon monoxide from methane, steam and CO₂ for the preparation of phosgene for synthesis of organic isocyanate, comprising at least the steps of
synthesis of carbon monoxide in a reformer process in which methane and steam are converted to carbon monoxide-containing product gas at a temperature of at least 500 °C with addition of at least CO₂ and with supply of thermal energy,
cleaning the carbon monoxide-containing product gas obtained from the above synthesis, at least by removal of CO₂ and optionally additionally by at least one removal selected from the removal of water, the removal of hydrogen or a combination thereof, to obtain carbon monoxide;
providing CO₂ for said addition to the aforementioned reformer process at least from said removal of CO₂ in the aforementioned cleaning step,
with the proviso that
1) the thermal energy supplied to the reformer process for the synthesis of carbon monoxide is provided by at least one method selected from (i) the combustion of fuel containing hydrogen produced by means of renewable energy, (ii) the combustion of fuel containing methane from a biological source, (iii) conversion of electrical energy generated from renewable energy to heat;
or
2) CO₂ is additionally supplied from a further CO₂ source for the provision of the CO₂ added to the synthesis in the aforementioned reformer process; or
3) a combination of the aforementioned options 1) and 2) is selected.

A useful source of methane for the reformer process is more preferably methane from a biological source. “Methane from a biological source” (also referred to as “biomethane”) is considered by the person skilled in the art to mean methane obtained industrially by methane fermentation from biomass, as opposed to fossil methane. Methane fermentation is known to mean the anaerobic degradation of organic substances by microorganisms. Methane from a biological source is produced, for example, in biogas plants in which both organic wastes and renewable raw materials are correspondingly fermented.

A further CO₂ source used for the abovementioned step 2) is preferably at least one external CO₂ source that contributes CO₂ which is not emitted by the process of the invention. An external CO₂ source would be, for example, the CO₂ which is obtained in cement production or in H₂ production for ammonia synthesis, is formed in the offgas on combustion of fuels (e.g. waste incineration), or CO₂ extracted from the air. This CO₂ from an external CO₂ source, in a preferred embodiment of the process of the invention, is effected by absorption of a CO₂ fraction from (i) process gases or offgases selected from at least one process selected from cement production, H₂ production, incineration, and/or (ii) from air by introduction into alkali metal hydroxide solution, for example potassium hydroxide solution. This results in the formation of potassium hydrogencarbonate, which can then be thermally decomposed back to CO₂ and potassium hydroxide. The CO₂ released is then fed to the reformer process for synthesis of carbon monoxide. Such released CO₂ can also, independently of the performance of a reformer process, additionally be fed to a reduction of CO₂ in A) (electrochemical reduction) or C) (RWGS reaction).

In a particularly preferred embodiment of the process of the invention, the carbon monoxide is prepared from said CO₂ for phosgene synthesis, especially for the synthesis of phosgene, by at least one reaction of hydrogen and CO₂ in a reverse water-gas shift (RWGS) reaction to give carbon monoxide. Such an embodiment is described, for example, in document WO 2021/089737 A, to which reference is made herein explicitly and in full. An embodiment of the process which is very particularly preferred in accordance with the invention is characterized in that the hydrogen stream is provided and converted together with the optionally cleaned CO₂ gas stream by the principle of the reverse water-gas shift reaction (especially in a reaction zone) to a product gas comprising carbon monoxide and possibly by-products.

A “reaction zone” is the part of a reaction space in which a chemical reaction, for example the reverse water-gas shift reaction, proceeds. A “reaction chamber” is a volume in which the coreactants taking part in a chemical reaction are brought together and in which the chemical reaction takes place. For a chemical reaction, this may, for example, be the volume of a vessel in which a reactant, for example carbon dioxide in the case of an RWGS reaction, and the coreactant thereof, hydrogen in the case of an RWGS reaction, are present together and are reacted in the reaction zone.

In this particularly preferred process, for production of carbon monoxide, very particular preference is given to performing at least the following steps:
feeding hydrogen gas provided together with said CO₂ gas stream into an RWGS reaction zone and reacting the reactants by the principle of the RWGS reaction to give a product gas mixture consisting of steam, CO and possibly by-products, in particular lower hydrocarbons, especially preferably methane;
separating unconverted carbon dioxide from the gas mixture of the RWGS reaction obtained from the separation, in particular by amine scrubbing, and recycling the unconverted carbon dioxide into the RWGS reaction;
separating the hydrogen unconverted in the RWGS reaction from the gas mixture of carbon monoxide and hydrogen obtained after the separation, in particular using a cold box, and optionally recycling the hydrogen into the RWGS reaction;
feeding the remaining carbon monoxide from the separation into the synthesis of phosgene.

The RWGS reaction is preferably conducted in the presence of at least one catalyst. The latter is more preferably selected from at least one compound from the group of:
(I) mixed metal oxides of the formula A_{(1-w-x)}A′_{w}A″ₓB_{(1-y-z)}B′_{y}B″_{z}O_{3-delta}
where:
A, A′ and A″ are independently selected from the group of: Mg, Ca, Sr, Ba, Li, Na, K, Rb, Cs, Sn, Sc, Y, La, Ce, Pr, Nd, Pm, Sm, Eu, Gd, Tb, Dy, Ho, Er, Tm, Yb, Tl, Lu, Ni, Co, Pb, Bi and/or Cd; and
B, B′ and B″ are independently selected from the group of: Cr, Mn, Fe, Bi, Cd, Co, Cu, Ni, Sn, Al, Ga, Sc, Ti, V, Nb, Ta, Mo, Pb, Hf, Zr, Tb, W, Gd, Yb, Mg, Li, Na, K, Ce and/or Zn; and
0≤w≤0.5; 0≤x≤0.5; 0≤y≤0.5; 0≤z≤0.5 and −1≤delta≤1;
(II) mixed metal oxides of the formula A_{(1-w-x)}A′_{w}A″ₓB_{(1-y-z)}B′_{y}B″_{z}O_{3-delta} where:
A, A′ and A″ are independently selected from the group of: Mg, Ca, Sr, Ba, Li, Na, K, Rb, Cs, Sn, Sc, Y, La, Ce, Pr, Nd, Sm, Eu, Gd, Tb, Dy, Ho, Er, Tm, Yb, Tl, Lu, Ni, Co, Pb and/or Cd; and
B is selected from the group of: Cr, Mn, Fe, Bi, Cd, Co, Cu, Ni, Sn, Al, Ga, Sc, Ti, V, Nb, Ta, Mo, Pb, Hf, Zr, Tb, W, Gd, Yb, Mg, Cd, Zn, Re, Ru, Rh, Pd, Os, Ir and/or Pt; and
B′ is selected from the group of: Re, Ru, Rh, Pd, Os, Ir and/or Pt; and B″ is selected from the group of: Cr, Mn, Fe, Bi, Cd, Co, Cu, Ni, Sn, Al, Ga, Sc, Ti, V, Nb, Ta, Mo, Pb, Hf, Zr, Tb, W, Gd, Yb, Mg, Cd and/or Zn; and
0≤w≤0.5; 0≤x≤0.5; 0≤y≤0.5; 0≤z≤0.5 and −1≤delta≤1;
(III) mixtures of at least two different metals M1 and M2 on a support comprising an oxide of Al, Ce and/or Zr that has been doped with a metal M3;
where: M1 and M2 are independently selected from the group of: Re, Ru, Rh, Ir, Os, Pd and/or Pt; and
M3 is selected from the group of: Sc, Y, La, Ce, Pr, Nd, Sm, Eu, Gd, Tb, Dy, Ho, Er, Tm, Yb and/or Lu;
(IV) mixed metal oxides of the formula LOₓ(M_{(y/z)}Al_{(2-y/z)}O₃)_{z}; where:
L is selected from the group of: Na, K, Rb, Cs, Mg, Ca, Sr, Ba, Sc, Y, Sn, Pb, Pd, Mn, In, Tl, La, Ce, Pr, Nd, Sm, Eu, Gd, Tb, Dy, Ho, Er, Tm, Yb and/or Lu; and
M is selected from the group of: Ti, Zr, Hf, V, Nb, Ta, Cr, Mo, W, Mn, Re, Fe, Ru, Os, Co, Rh, Ir, Ni, Pd, Pt, Zn, Cu, Ag and/or Au; and
1<x≤2; 0<y≤12; and 4≤z≤9;
(V) mixed metal oxides of the formula LO(Al₂O₃)_{z}; where:
L is selected from the group of: Na, K, Rb, Cs, Mg, Ca, Sr, Ba, Sc, Y, Sn, Pb, Mn, In, Tl, La, Ce, Pr, Nd, Sm, Eu, Gd, Tb, Dy, Ho, Er, Tm, Yb and/or Lu; and
4≤z≤9;
(VI) oxidic catalyst comprising Ni and Ru;
(VII) metal M1 and/or at least two different metals M1 and M2 on and/or in a support, where the support
is a carbide, oxycarbide, carbonitride, nitride, boride, silicide, germanide and/or selenide of metals A and/or B; where:
M1 and M2 are independently selected from the group of: Cr, Mn, Fe, Co, Ni, Re, Ru, Rh, Ir, Os, Pd, Pt, Zn, Cu, La, Ce, Pr, Nd, Sm, Eu, Gd, Tb, Dy, Ho, Er, Tm, Yb and/or Lu; and
A and B are independently selected from the group of: Be, Mg, Ca, Sc, Ii, V, Cr, Mn, Fe, Co, Ni, Y, Zr, Nb, Mo, Hf, Ta, W, La, Ce, Pr, Nd, Sm, Eu, Gd, Tb, Dy, Ho, Er, Tm, Yb and/or Lu;
and/or
reaction products of (I), (II), (III), (IV), (V), (VI) and/or (VII) in the presence of carbon dioxide, hydrogen, carbon monoxide and/or water at a temperature of ≥700 °C.

The hydrogen stream provided for the RWGS reaction, in a further embodiment, is preferably obtained by electrolysis of water to give hydrogen and oxygen. Water electrolysis for production of hydrogen gas is the electrochemical conversion of water. Water is split here into hydrogen and oxygen using electricity. Known processes are proton exchange electrolysis (PEM electrolysis), which is described, for example, in EP 3489394 A. A further process is alkaline water electrolysis. The two processes differ in particular by the process parameters, for example temperature, pressure and pH. However, both processes lead to the target products of water and oxygen gas. Water electrolysis can be carried out with prior art plants. Industrial systems for alkaline water electrolysis and also for PEM electrolysis are known and commercially available. The principles of water electrolysis are described by way of example in chapter 6.3.4 in Volkmar M. Schmidt in “Elektrochemische Verfahrenstechnik” [Electrochemical process technology] (2003 Wiley-VCH-Verlag; ISBN 3-527-29958-0).

It is particularly preferable when the water electrolysis (5), for provision of the hydrogen stream, is performed using electricity generated from renewable energy, in particular from renewable energy in the form of wind power, solar power or hydropower.

The carbon monoxide obtained from the reduction of CO₂, if necessary, is cleaned to free it of any by-products present and used for synthesis of phosgene. This synthesis of phosgene is by reaction of at least chlorine gas and carbon monoxide, where the carbon monoxide reacted is at least the carbon monoxide from the reduction of CO₂ and phosgene is discharged.

The cleaning of the carbon monoxide is preferably effected prior to use in the synthesis of phosgene by at least the following steps:
removal of water,
removal of unconverted carbon dioxide from the gas mixture obtained from the removal of water, especially by amine scrubbing and recycling of the unconverted carbon dioxide into the reduction of CO₂ in the process of the invention,
removal of any hydrogen present from the gas mixture obtained after the removal of unconverted carbon dioxide, especially using a cold box.

In a further preferred embodiment of the process, carbon dioxide is separated off after water has been separated from the carbon monoxide-containing product gas beforehand. For this purpose, the carbon monoxide-containing gas is first fed to a water removal unit, where the water is separated off, and the carbon monoxide-containing, dry product gas obtained after the separation of water is fed to a CO₂ removal unit in which the CO₂ is separated off. In the water removal unit, the water is separated off, for example, by cooling the carbon monoxide-containing gas and separating off the water, for example as condensate.

What is meant here in particular by “amine scrubbing” is fundamentally known scrubbing by the principle of chemisorption with amines such as monoethanolamine (MEA), diethanolamine (DEA), methyldiethanolamine (MDEA) or diglycolamine (DGA), which even at relatively low pressure in an absorption column achieves a high purity of the purified gas mixture.

A variant of the process which is preferred in accordance with the invention is a process in which the carbon monoxide-containing gas (preferably having been freed of water and of CO₂ beforehand) is introduced into an H₂—CO separation unit in which hydrogen is separated off. This forms at least one gas stream, wherein the gas at 25 °C and 1013 mbar contains at least 95% by volume of carbon monoxide, more preferably at least 99% by volume of carbon monoxide. Said introduced product gas is preferably first separated into two gas streams in the H₂—CO separation unit. This gives rise to a gas in the form of a gas stream containing at least 95% by volume (preferably at least 99% by weight) of carbon monoxide, a further gas in the form of a gas stream having hydrogen as its major constituent and carbon monoxide and methane inter alia. The further gas is also referred to as tail gas from the H₂—CO separation or, if there is no tail gas treatment, as end gas. A H₂—CO separation unit that works by this principle of separation is called the coldbox. The hydrogen-containing tail gas from the H₂—CO separation can be subjected to a subsequent tail gas treatment to concentrate the hydrogen. Processing by tail gas treatment affords a gas enriched with hydrogen gas in the form of a gas stream and another gas—called the tail gas from the tail gas treatment or end gas—in the form of a gas stream comprising a mixture of CO, methane and a smaller amount of hydrogen than the hydrogen gas-enriched gas.

For the provision of the chlorine for the synthesis of phosgene effective in this embodiment, those skilled in the art will be well acquainted with the production of chlorine gas from electrochemical oxidation by the electrolysis of hydrochloric acid with a gas-diffusion electrode (also referred to as the HCl ODC electrolysis process (ODC=oxygen-depleting electrode); for a suitable electrolysis cell see U.S. Pat. No. 6,022,634 A, WO 03/31690 A1), the production of chlorine gas from hydrochloric acid diaphragm electrolysis (see EP 1 103 636 A1), the production of chlorine gas from thermocatalytic gas-phase oxidation (see WO 2012/025483 A2), and the production of chlorine from chloralkali electrolysis (see WO 2009/007366 A2). Reference is made expressly and in full to the content of the abovementioned documents cited in connection with the production of chlorine gas. The chlorine required for the synthesis of phosgene is in a preferred variant of this embodiment of the process produced electrolytically, especially through electrochemical oxidation by hydrochloric acid electrolysis with a gas-diffusion electrode, through electrochemical oxidation by hydrochloric acid diaphragm electrolysis or through electrochemical oxidation by chloralkali electrolysis. It is in this case in turn particularly preferable when said electrochemical oxidation is in each case carried out using electricity generated from renewable energy, especially from renewable energy in the form of wind power, solar energy or hydro power.

The phosgene formed by the phosgene synthesis, in a particularly preferred embodiment of the process, is used in a further step for production of organic isocyanate, in which the phosgene from the phosgene synthesis is reacted with at least one organic amino compound, especially with organic amino compound obtained in the hydrolysis of the organically modified carbamate, and at least organic isocyanate is discharged. Preferably, at least organic isocyanate and hydrogen chloride are discharged.

In general, preference is in this embodiment given to those processes of the invention in which the organic isocyanate obtained contains at least two isocyanate groups. For this purpose, the reactant used in the synthesis is again preferably organic amino compound having at least two amino groups, especially the organic amino compound obtained in the hydrolysis of the organically modified carbamate.

More preferably, the organic isocyanate obtained contains at least two isocyanate groups and has a molar mass of not more than 1000 g/mol, especially of not more than 800 g/mol.

Organic amines used with very particular preference are selected from tolylenediamine (TDA), methylenedi (phenylamine) (MDA) (preferably selected in turn from diphenylmethane-2,2′-diamine, diphenylmethane-2,4′-diamine, diphenylmethane-4,4′-diamine or mixtures thereof), hexamethylenediamine, isophoronediamine, 1,3-bis(aminomethyl)benzene, cyclohexyldiamine or mixtures thereof, where TDA, MDA or mixtures thereof, especially the TDA or MDA obtained in the hydrolysis of the organically modified carbamate, are very particularly preferred organic isocyanates.

Organic isocyanates obtained with very particular preference are selected from tolylene diisocyanate (TDI), methylene di(phenyl isocyanate) (MDI) (preferably selected in turn from diphenylmethane 2,2′-diisocyanate, diphenylmethane 2,4′-diisocyanate, diphenylmethane 4,4′-diisocyanate or mixtures thereof), hexamethylene diisocyanate (HDI), isophorone diisocyanate (IPDI), 1,3-bis(isocyanatomethyl)benzene (XDI), cyclohexyl diisocyanate (CHDI) or mixtures thereof, where TDI, MDI or mixtures thereof are very particularly preferred organic isocyanates.

Furthermore, in the embodiment with synthesis of organic isocyanate it is preferable that hydrogen chloride is additionally formed alongside said organic isocyanate. It is again preferable to feed in this hydrogen chloride as reactant in the preparation of chlorine for said synthesis of phosgene. The necessary removal and purification of the hydrogen chloride formed in the production of isocyanate can then be accomplished through oxidative reaction in a thermocatalytic gas-phase oxidation with oxygen to chlorine and water, and optionally using O₂ from water electrolysis.

A further alternative approach to utilizing the hydrogen chloride as a reactant for the production of chlorine is the reaction of the hydrogen chloride with water to hydrochloric acid and subsequent electrochemical oxidation of the hydrochloric acid to chlorine and optionally hydrogen. This aqueous hydrochloric acid is in particular supplied to the previously described electrochemical oxidation by hydrochloric acid electrolysis with a gas-diffusion electrode, electrochemical oxidation by hydrochloric acid diaphragm electrolysis or electrochemical oxidation by chloralkali electrolysis). O₂ required in the case of hydrochloric acid electrolysis with a gas-diffusion electrode can be obtained from water electrolysis.

The hydrogen chloride resulting from isocyanate production can also, after purification and absorption in water, be converted to hydrochloric acid and the resulting hydrochloric acid sold on the market for various uses.

In a particularly preferred embodiment of the process, the following are additionally performed:
a phosgene synthesis by reaction of at least chlorine gas and the carbon monoxide obtained previously after cleaning of said carbon monoxide-containing product gas from step c);
an isocyanate preparation by reaction of the phosgene obtained with at least one organic amine to form at least one organic isocyanate compound and hydrogen chloride;
a separation and purification of the hydrogen chloride formed in the production of isocyanate and a subsequent oxidative reaction of the hydrogen chloride, selected from
   a reaction of hydrogen chloride in a thermocatalytic gas-phase oxidation with oxygen to chlorine and water, optionally using oxygen from water electrolysis,
   a reaction of hydrogen chloride with water to form hydrochloric acid and subsequent electrochemical oxidation of hydrochloric acid to chlorine and optionally hydrogen;
and, following the oxidative conversion of the hydrogen chloride, feeding of the chlorine formed to said synthesis of phosgene, optionally while supplying said synthesis of phosgene with an additional amount of chlorine that comes from a chloralkali electrolysis.

The organic isocyanates obtained may optionally, in an additional step of the process, be reacted with at least one organic compound having at least two hydroxy groups, especially at least one polyester polyol or polyether polyol, to form polyurethane material.

Correspondingly produced polyurethane materials are, for example, in the form of foams, coatings, insulating compound and components of commercial products. They may be provided again at the end of their life cycle as polyurethane material for the process of the invention.

The invention further provides for the use of CO₂, which is the direct product of a process comprising at least the step of hydrolysis of at least one organically modified carbamate, in a reduction reaction (i.e. a reduction of CO₂) for production of carbon monoxide for phosgene production.

Corresponding embodiments of the process of the invention are also applicable mutatis mutandis to this subject of the invention.

The invention still further provides for the use of carbon monoxide, which is the direct product of a process comprising at least a reduction reaction of CO₂ that has been prepared by hydrolysis of at least one organic compound selected from organically modified carbamate, for production of phosgene.

Corresponding embodiments of the process of the invention are also applicable mutatis mutandis to this subject of the invention.

The invention still further provides an apparatus for production of phosgene, comprising
at least one unit for provision of a CO₂ gas stream, which is the direct product of a process comprising at least the step of hydrolysis of at least one organic compound selected from organically modified carbamate, where this unit comprises at least one CO₂ outlet for said CO₂ gas stream; and
optionally at least one unit for cleaning of the CO₂ gas stream to remove secondary constituents, comprising at least one unit from the group formed by condensation unit, adsorption unit, gas scrubbing unit or catalytic gas cleaning unit, where the unit for cleaning of the CO₂ gas stream has at least one inlet for the CO₂ gas stream as CO₂ gas stream to be cleaned, which is fluidically connected to the CO₂ outlet of the unit for provision of the CO₂ gas stream, and where the unit for cleaning of the CO₂ gas stream has at least one outlet for a cleaned CO₂ gas stream; and
at least one unit for reduction of the at least one optionally cleaned CO₂ gas stream provided to carbon monoxide, comprising at least one inlet for the CO₂ gas stream which is fluidically connected to at least one outlet of the unit for provision of a CO₂ gas stream, optionally via the at least one unit for cleaning of the CO₂ gas stream, and comprising at least one outlet for carbon monoxide;
at least one unit for production of phosgene, comprising
   at least one inlet for chlorine gas, fluidically connected to a source for chlorine gas, and
   at least one inlet for carbon monoxide, fluidically connected to at least one outlet for carbon monoxide in the unit for reduction of said CO₂ gas stream;
   at least one outlet for phosgene.

A fluid connection is understood to mean an apparatus which connects apparatus components to one another and through which a substance that may be in any state of matter can be transported as a stream of matter from one apparatus component to the next, for example an inlet in the form of a pipe. The expression “are in fluid connection” means that named apparatus components are connected to one another via a fluid connection.

A “reaction space” is a volume of an apparatus in which the co-reactants taking part in a chemical reaction are present together and in which the chemical reaction takes place. For a chemical reaction, this may be, for example, the volume of a vessel or container in which the co-reactants are present together and are to be reacted.

A preferred embodiment of the apparatus is suitable for the simultaneous execution of the chemolytic conversion and the hydrolysis in one step and is characterized in that the unit for provision of a CO₂ gas stream comprises a unit for hydrolysis of at least one organic compound, comprising
at least one hydrolysis apparatus comprising at least one inlet for a chemolysis reagent, at least one inlet for water-containing liquid, at least one inlet for polyurethane material and the at least one outlet for the CO₂ gas stream, where said organic substance and said liquid can be contacted in a reaction zone of the hydrolysis apparatus, and the CO₂ gas stream formed can be conducted out via said outlet.

A further preferred embodiment of the apparatus is suitable for the separate execution of the chemolytic conversion and the hydrolysis and is characterized in that the unit for provision of a CO₂ gas stream comprises a unit for hydrolysis of at least one organic compound, comprising
at least one chemolysis apparatus comprising at least one inlet for a chemolysis reagent-containing liquid, at least one inlet for polyurethane material and the at least one outlet for organically modified carbamate, where said organic substance and said liquid can be contacted in the reaction zone of the reactor and converted to at least one organic compound selected from organically modified carbamate; and
at least one hydrolysis apparatus comprising at least one inlet for a water-containing liquid, at least one inlet for organically modified carbamate and the at least one outlet for the CO₂ gas stream, where said organically modified carbamate and said water-containing liquid can be contacted in a reaction zone of the hydrolysis apparatus, and the CO₂ gas stream formed can be conducted out via said outlet, where the inlet for organically modified carbamate is fluidically connected to the outlet for organically modified carbamate in the reactor for chemolysis.

It is further preferable in the context of this embodiment when the unit for hydrolysis of at least one organic compound additionally comprises at least one separation apparatus, where the outlet for organically modified carbamate from the chemolysis apparatus is fluidically connected to the inlet of the separation apparatus, and the separation apparatus has at least one inlet for organic solvent which is incompletely miscible with the chemolysis reagent used beforehand, which is fluidically connected to a source of said organic solvent and contains at least one outlet for a carbamate phase which is fluidically connected to the inlet of the hydrolysis apparatus. The separation apparatus is configured such that the composition obtained in the chemolysis, at least comprising at least one organically modified carbamate, can be contacted with an organic solvent which is incompletely miscible with the chemolysis reagent used beforehand, and, after mixing and contacting, a phase separation can be brought about into a carbamate phase (containing organically modified carbamate) and a solvent phase, from which the carbamate phase can be separated and discharged from the separation apparatus. In this embodiment, the carbamate phase is introduced into the hydrolysis apparatus.

In a further preferred embodiment of the apparatus, the unit for reduction of the at least one optionally cleaned CO₂ gas stream (31, 31a) provided is an electrolysis unit with a gas diffusion electrode for CO₂ reduction or a reverse water-gas shift reactor.

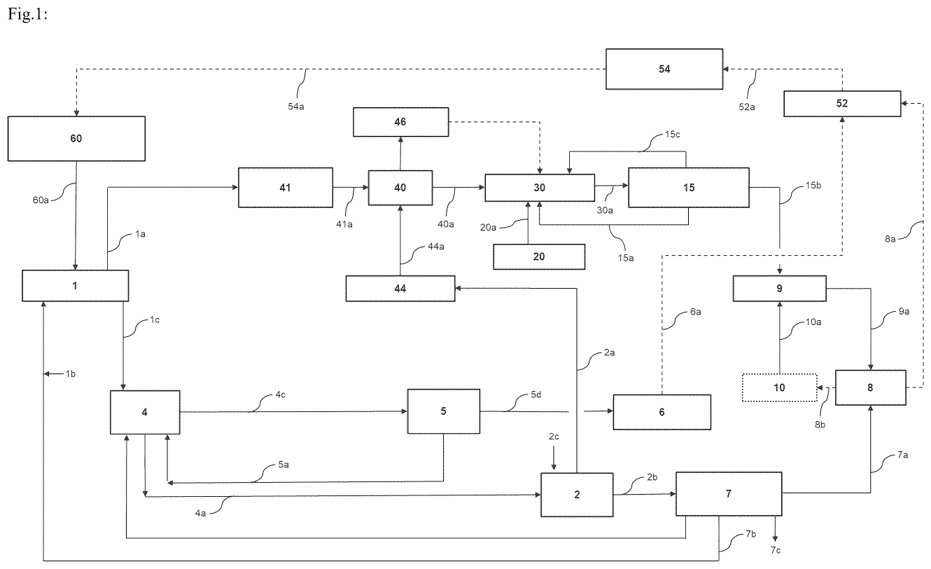

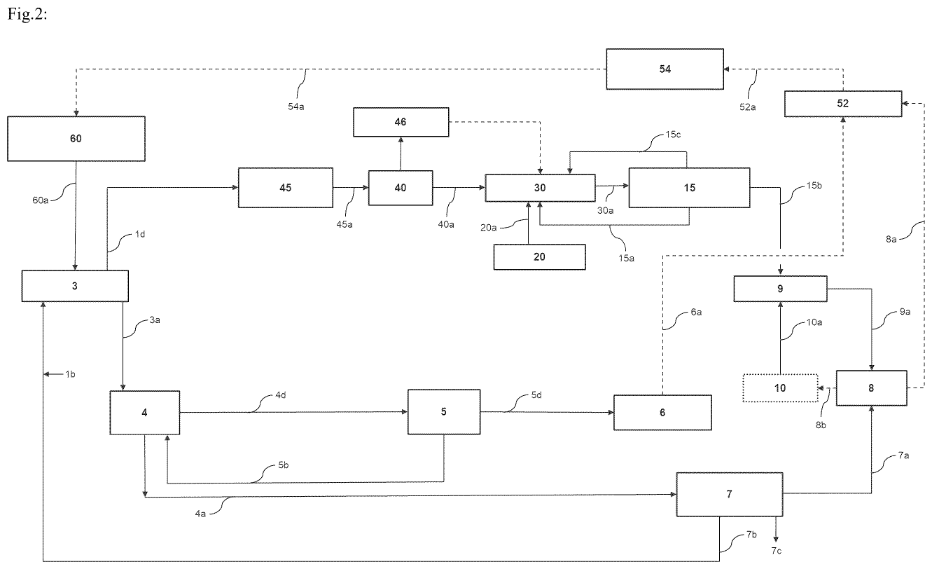

The invention is elucidated in detail hereinafter by examples 1 to 4 with reference to FIGS. 1 and 2, without restricting the invention to these examples. In FIGS. 1 and 2, the following reference numerals have the definition on the right in each case:
1 chemolysis, here: glycolysis
1a gas stream of CO₂ from chemolysis (glycolysis)
1b chemolysis reagent (here: diethylene glycol (DEG)) addition to replace losses
1c polyol from chemolysis
1d gas stream of CO₂ from coupled chemolysis with hydrolysis (here: hydroglycolysis)
1e H₂O+catalyst Na₂CO₃ for coupled chemolysis and hydrolysis (here: hydroglycolysis)
2 hydrolysis
2a gas stream of CO₂ from hydrolysis
2b TDA from hydrolysis
2c H₂O for hydrolysis
3 coupled chemolysis with hydrolysis (here: hydroglycolysis)
3a polyol solvent from hydroglycolysis
4 solvent extraction
4a organic amino compound (here: TDA)/organically modified carbamates from solvent extraction
4b H₂O replacement of losses
4c polyol toluene from solvent extraction
4d polyol and solvent from solvent extraction
5 solvent evaporation
5a toluene from solvent evaporation
5b cyclohexane solvent from 5
5c solvent replacement for losses
5d polyol
6 polyol production
6a polyol from polyol production
6b discharge of residues
7 amine cleaning
7a organic amino compound (here aTDA) from amine cleaning
7b chemolysis reagent (here DEG) from amine cleaning
7c residue
7d H₂O from amine cleaning
7f H₂O from amine purification
8 isocyanate production
8a TDI
8b HCl from isocyanate production
9 phosgene production
9a phosgene
10 HCl recycling
10a chlorine from HCl recycling
11 PU foam production
15 CO process gas processing by: cooling, drying, CO₂ removal (amine scrub) & H₂—CO separation
15a H₂ from the H₂—CO separation
15b CO from the H₂—CO separation
15c CO₂ from the CO₂ removal
20 H₂ production
20a H₂ from water electrolysis
20c H₂O for replacement of water electrolysis losses
30 CO production by reduction of CO₂ (CO₂ electrolysis or RWGS)
30a CO product gas stream from the reduction of CO₂
40 CO₂ compression
40a CO₂ after compression
41 CO₂ processing (CO₂ from chemolysis)
41a CO₂ from chemolysis after processing
41b CO₂ purge stream from 41
41c CO₂ purge stream from 44
44 CO₂ processing from hydrolysis
44a CO₂ purified from hydrolysis
45 CO₂ hydroglycolysis workup
45a CO₂ purified from hydroglycolysis workup
45b CO₂ purge from workup 45
46 CO₂ reservoir (optional)
52 production of polyurethane material (here: PU foam production)
52a polyurethane material for the market
54 market/valuable material collection/separation
54a recycled PU foam
60 provision of polyurethane material (here: PU foam preparation & inertization)
60a polyurethane material (here: TDI-based PU foam)
70 RWGS heating—electrical or biogas or H₂

### EXAMPLES

### Example 1 (FIG. 1)

300 kg of a polyurethane material 60a in the form of a residual moisture-containing polyurethane foam having a proportion by weight of 33% by weight of TDI and 67% by weight of polyol (ARCOL® Polyol 1108) is provided. This is dissolved with 300 kg of diethylene glycol as chemolysis reagent with addition of 5.5 kg of Na₂CO₃ as catalyst in a chemolysis 1 under an inert carbon dioxide gas atmosphere. The product mixture obtained in the dissolution, with 227.1 kg of polyol, 251.1 kg of diethylene glycol and 119.6 g of TDA/organically modified carbamates, prior to hydrolysis 2, is introduced into a solvent extraction 4 with toluene as solvent, which is conducted analogously to the example from document WO 2020/260387 A1. The solvent phase obtained after the phase separation, with 1367 kg of toluene and 227.1 kg of polyol, is transferred into a solvent evaporation 5, where the toluene solvent is separated virtually completely from the polyol and recycled into the solvent extraction 4. The resultant polyol is subjected to further workup in polyol production 6.

The carbamate phase obtained after phase separation in the solvent extraction 4, containing 119.6 kg of TDA/organically modified carbamate, is transferred into the hydrolysis 2, where it is hydrolyzed in an inert carbon dioxide gas atmosphere with addition of a total of 16.5 kg of water at 180 °C for 2.5 h. Overall, a liquid composition with 51.1 kg of TDA and 300 kg of diethylene glycol, and a CO₂ gas stream 2a are obtained. The liquid composition is transferred into an amine cleaning operation 7, where it is separated into 47.92 kg of TDA 7a and about 300 kg of diethylene glycol 7b and residual material 7c.

A gas stream 1a with 3.7 kg/h of CO₂, containing traces of N₂ and O₂, DEG, water, is withdrawn from the chemolysis 1 and sent to the CO₂ workup 41. The gas stream 1a has a temperature of 180 °C and a pressure of 1.01 bar_{(abs)}. The gas stream 1a is cooled down to 25 °C by means of heat exchangers, and in so doing largely freed of water and DEG.

Subsequently, the gas stream 1a is dried via zeolite, a substream 41a is purged with 0.3 kg of CO₂, and the tail gas stream 41a with 3.4 kg of CO₂ is sent to the compression operation 40 at 25 °C.

Withdrawn from the hydrolysis 2 is a CO₂ gas stream 2a with 33.2 kg/h of CO₂ and traces of N₂ and O₂, DEG, water, toluene. The gas stream has a temperature of 180 °C and a pressure of 1.01 bar_{(abs)}. The gas stream 2a is cooled down to 25 °C by means of heat exchangers, and in so doing largely freed of water and DEG.

Subsequently, the cooled gas stream is dried via zeolite, a substream is purged with 2 kg/h of CO₂, and the tail gas stream 44a with 31.2 kg/h of CO₂ is sent to the compression operation 40 at 25 °C.

A continuous gas stream 40a with 34.6 kg/h of CO₂ at 30 bar and 40 °C is withdrawn from the compressor stage 40, and sent to a CO production operation by reduction of CO₂ 30 in the form of an RWGS into the reaction space of the RWGS.

The RWGS reaction is run at 802 °C, with maintenance of the reaction temperature by introduction and combustion of bio-natural gas. It would optionally also be possible to use H₂ for heating or to use electrical heating. 34.6 kg/h of CO₂ and 1.57 kg/h of H₂ 20a from a water electrolysis 20 are introduced into the RWGS reaction space of the CO production by reduction of CO₂ 30, which is run at a temperature of 802 °C and 30 bar.

The resultant product gas mixture 30a consisting of CO, H₂O, unconverted CO₂ and unconverted H₂, and also by-products, mainly small amounts of methane, are withdrawn from the RWGS reaction and cooled down in a process gas cooling operation of the CO process gas processing operation 15.

Thereafter, the gas mixture is sent to a process gas drying operation in the CO process gas processing operation 15, in which a total of about 14.1 kg/h of water is removed. This water is sent to the water electrolysis 20.

A total of 1.57 kg/h of hydrogen is removed from the water electrolysis 20, and 14.13 kg/h of water is fed to the water electrolysis. This water is composed of the recycled water from the process gas drying operation and freshly added water.

The remaining gas mixture from the process gas drying operation is sent to a CO₂ removal operation in the CO process gas processing operation 15. The CO₂ is removed by amine scrubbing, and the removed CO₂ 15c is fed back to the reduction of CO₂ 30 (RWGS reaction).

The energy for CO₂ removal from the CO₂-amine complex formed is sourced from the abovementioned process gas cooling operation in which the RWGS reaction gases gases are cooled down. The gas freed of CO₂ is sent to the H₂—CO separation removal in the CO process gas processing operation 15. A cold box is used for the H₂—CO separation, in which the H₂—CO gas mixture is cooled down and hydrogen and CO are separated. The removed hydrogen (15a) is fed back to the RWGS reaction (31). 22.0 kg/h of CO 15b from the H₂—CO separation is sent to a phosgene synthesis 9. In the latter, the CO 15b reacted with 55.78 kg/h of chlorine 10a that was taken from an HCl recycling process 10. The HCl gas is converted to chlorine by thermocatalytic gas phase oxidation (Deacon) of HCl from isocyanate production 8. Optionally, the O₂ from the water electrolysis 20 can be used for the oxidation of HCl gas in the Deacon process 10.

77.78 kg/h phosgene is withdrawn from the phosgene synthesis 9 and reacted in isocyanate production 8 with 47.92 kg/h of toluenediamine 7a to give 68.35 kg/h of toluene diisocyanate 8a.

The resultant HCl gas in an amount of 57.35 kg/h, after cleaning via a low-temperature distillation, is sent to Cl₂ production by thermocatalytic gas phase oxidation 10. In the thermocatalytic gas phase oxidation 10, the HCl gas is reacted with oxygen at about 300 °C over a ruthenium oxide-based catalyst to give chlorine and H₂O. The oxygen required (12.57 kg/h) is withdrawn from the water electrolysis 20.

The resultant toluene diisocyanate (68.35 kg/h) 8a is reacted again with the 227.1 kg/h of polyol (corresponding essentially to the specification of ARCOL® Polyol 1108) 6a obtained from the process to give 295.44 kg/h of polyurethane material in PU foam production 52.

After the polyurethane material has been used in various applications on the market 54, it can be collected and recycled in order to provide the polyurethane material obtained therefrom as material of value to the chemolysis (1) after treatment and processing 60.

The hydrogen 20a is generated in a water electrolysis 20 with a power of 0.088 MW using renewable energy. The water electrolysis 20 is an alkaline water electrolysis operated with a current density of 8 kA/m² and a cell voltage of 2 V per electrolysis element. 0.088 MW and 14.13 kg/h of water (16a+20c) are supplied here from the process gas cooling operation and the process gas drying operation, in each case from the removal from the CO process gas processing operation 15, and externally. 1.57 kg/h of H₂ and 12.56 kg/h of oxygen are withdrawn from the water electrolysis.

By virtue of the process of the invention, all raw materials of the TDI-based PU material, such as the amine and the polyol, are recycled virtually completely and hence the value creation cycle is completed.

The use of renewable energy in water electrolysis further reduces the CO₂ footprint of the phosgene produced from CO and Cl₂ and hence enables greater sustainability of production of TDI or of the resulting PU material.

### Example 2 (FIG. 1)

Apart from the execution of the CO₂ reduction, this example corresponds to example 1.

A low-temperature CO₂ electrolysis is performed as the reduction of CO₂ 30. The CO₂ electrolysis is appropriately operated in accordance with European patent application number 18195279.7, example 1. The electrolysis is run at a pressure of 1.2 bar_{(abs)}. 10 elements each of electrode area 1.6 m² are used, which are connected together to form an electrolyzer. The electrolysis is operated at a cell voltage of 3.7 V and a current density of 4.05 kA/m², with a current yield with respect to CO of 65%, 0.239 MWh of renewable energy, especially wind power, is consumed, corresponding to a terminal power of 0.239 MW.

34.6 kg/h of CO₂ is fed to the CO₂ electrolysis 30 at a pressure of 1.2 bar_{(abs)} after drying, cleaning in 41 and compression 40. A gas mixture 30a of 22 kg/h of CO, 200 kg/h of CO₂, 0.85 kg/h of H₂ is withdrawn from the CO₂ electrolysis 30.

In addition, 19.36 kg/h of O₂ is withdrawn from the anode space.

The gas mixture 30a produced from the electrolysis 30 is sent to a process gas processing operation 15 with a drying operation & a CO₂ removal operation in the form of an amine scrub, and the unconverted CO₂ 15c is separated from the mixture and returned to the electrolysis 30. The gas freed of CO₂, consisting of CO and H₂, is fed to a CO—H₂ separation in the form of a cold box, in which CO and H₂ are separated. 22 kg/h of CO 15b is reacted with the Cl₂ 10a coming from the HCl recycling 10 to give phosgene 9a in the phosgene production 9, and this is reacted with the TDA 7a coming from the hydrolysis 2 and the amine cleaning 7 to give TDI 8a in the isocyanate production 8.

### Example 3 (FIG. 2)

300 kg of a polyurethane material 60a in the form of a residual moisture-containing polyurethane foam having a proportion by weight of 33% by weight of TDI and 67% by weight of polyol (ARCOL® Polyol 1108) is provided. This is reacted with 300 kg of diethylene glycol as chemolysis reagent with addition of 5.5 kg of Na₂CO₃ as catalyst in a combined chemolysis and hydrolysis (here: hydroglycolysis) 3 with a total of 16.5 kg of water under an inert carbon dioxide gas atmosphere analogously to the manner described in example x of document WO XXX. A product solution 3a and a CO₂ gas stream 1d are obtained.

After the reaction, the product solution is transferred into a solvent extraction 4 and mixed with 1866 kg of cyclohexane. The solvent phase 4d obtained after the phase separation, with 1866 kg of cyclohexane, 223.6 kg of polyol and 1.8 kg of residual material, is transferred into a solvent evaporation 5, where the cyclohexane solvent is separated virtually completely from the polyol and recycled into the solvent extraction 4. The resultant polyol is subjected to further workup in the polyol production 6 and separated from the residual materials 6b.

The amine-containing phase (TDA) obtained in the solvent extraction 4 after the phase separation, containing 52 kg of TDA water, salts and 300 kg of DEG is transferred into an amine cleaning operation 7, where it is separated into 47.92 kg of TDA 7a and about 300 kg of diethylene glycol 7b and residual material 7c.

A gas stream 1d with 37.4 kg/h of CO₂, containing traces of N₂ and O₂, DEG, water, is withdrawn from the hydroglycolysis 3 and fed to the CO₂ workup 45. The gas stream 1d has a temperature of 180 °C and a pressure of 1.01 bar_{(abs)}. The gas stream 1d is cooled down to 25 °C by means of heat exchangers, and in so doing largely freed of water and DEG.

Subsequently, the gas stream 1d is dried via zeolite, a substream 45b is purged with 2.8 kg of CO₂, and the tail gas stream 45a with 34.6 kg/h of CO₂ is sent to the compression operation 40 at 25 °C.

A continuous gas stream 40a with 34.6 kg/h of CO₂ at 30 bar and 40 °C is withdrawn from the compressor stage 40, and sent to a CO production operation by reduction of CO₂ 30 in the form of an RWGS into the reaction space of the RWGS.

The RWGS reaction 30 is run at 802 °C, with maintenance of the reaction temperature by introduction and combustion of bio-natural gas. It would optionally also be possible to use H₂ for heating or to use electrical heating. 34.6 kg/h of CO₂ 40a and 1.57 kg/h of H₂ 20a from a water electrolysis 20 are introduced into the RWGS reaction space 30, which is run at a temperature of 802 °C and 30 bar.

The resultant product gas mixture 30a consisting of CO, H₂O, unconverted CO₂ and unconverted H₂, and also by-products, mainly small amounts of methane, are withdrawn from the RWGS reaction and cooled down in a process gas cooling operation of the process gas processing operation 15.

Thereafter, the gas mixture is sent to a process gas drying operation process gas processing operation 15, in which a total of about 14.1 kg/h of water is removed. This water is sent to the water electrolysis 20.

A total of 1.57 kg/h of hydrogen 20a is removed from the water electrolysis 20 and 14.13 kg/h of water is fed to the water electrolysis. This water is composed of the recycled water from the abovementioned process gas drying operation and freshly added water.

The remaining gas mixture from the process gas drying operation is fed to a CO₂ removal operation process gas processing operation 15. The CO₂ is removed by amine scrubbing, and the removed CO₂ 15c is fed back to the RWGS reaction 30.

The energy for CO₂ removal from the CO₂-amine complex formed is sourced from the process gas cooling operation in the process gas processing operation 15 in which the RWGS reaction gases gases 30a are cooled down. The gas freed of CO₂ is sent to the H₂—CO separation in the process gas processing operation 15. A cold box is used for the H₂—CO separation, in which the H₂—CO gas mixture is cooled down and hydrogen and CO are separated. The removed hydrogen 15a is fed back to the RWGS reaction 30. 22.0 kg/h of CO 15b from the H₂—CO separation is sent to a phosgene synthesis 9. In the latter, the CO 15b reacted with 55.78 kg/h of chlorine 10a that was taken from an HCl recycling process 10. The HCl gas is converted to chlorine by thermocatalytic gas phase oxidation (Deacon) of HCl from isocyanate production 8. Optionally, the O₂ from the water electrolysis 20 can be used for the oxidation of HCl gas in the Deacon process 10.

77.78 kg/h phosgene is withdrawn from the phosgene synthesis 9 and reacted in isocyanate production 8 with 47.92 kg/h of toluenediamine 7a to give 68.35 kg/h of toluene diisocyanate 8a.

The resultant HCl gas in an amount of 57.35 kg/h, after cleaning via a low-temperature distillation, is sent to Cl₂ production by thermocatalytic gas phase oxidation (10). In the thermocatalytic gas phase oxidation 10, the HCl gas is reacted with oxygen at about 300 °C over a ruthenium oxide-based catalyst to give chlorine and H₂O. The oxygen required (12.57 kg/h) is withdrawn from the water electrolysis 20.

The resultant toluene diisocyanate (68.35 kg/h) 8a is reacted again with the 223.6 kg/h of polyol (corresponding essentially to the specification of ARCOL® Polyol 1108) 6a obtained from the process to give 291.95 kg/h of polyurethane material in PU foam production 52.

After the polyurethane material has been used in various applications on the market 54, it can be collected and recycled in order to provide the polyurethane material waste obtained therefrom to the hydroglycolysis 3 after treatment and processing 60.

The hydrogen 20a is generated in a water electrolysis 20 with a power of 0.088 MW using renewable energy. The water electrolysis 20 is an alkaline water electrolysis operated with a current density of 8 kA/m² and a cell voltage of 2 V per electrolysis element. 0.088 MW and 14.13 kg/h of water are supplied here from the process gas cooling operation in the process gas processing (PGP) operation 15, the process gas drying operation of the PGP 15 and externally. 1.57 kg/h of H₂ and 12.56 kg/h of oxygen are withdrawn from the water electrolysis.

By virtue of the process of the invention, all raw materials of the TDI-based PU material, such as the amine and the polyol, are recycled virtually completely and hence the value creation cycle is completed.

The use of renewable energy in water electrolysis further reduces the CO₂ footprint of the phosgene produced from CO and Cl₂ and hence enables sustainable produced TDI or the resulting PU material.

### Example 4 (FIG. 2)

Apart from the execution of the CO₂ reduction, this example corresponds to example 3.

A low-temperature CO₂ electrolysis as described in example 2 is performed as the reduction of CO₂ 30.

1. A process for producing phosgene, the process comprising:
producing at least one CO₂ gas stream by at least the following steps:
providing at least one organically modified carbamate;
hydrolyzing the at least one organically modified carbamate provided to form at least organic amino compound and CO₂; and
separating off the CO₂ formed to obtain the at least one CO₂ gas stream;
reducing CO₂ in the at least one CO₂ gas stream to carbon monoxide; and
synthesizing phosgene at least from the carbon monoxide obtained by the reduction and chlorine.

2. The process according to claim 1, wherein the at least one organically modified carbamate provided is at least one direct process product of a chemolysis of a polyurethane material, comprising at least the following process steps:
providing the polyurethane material; and
reacting the polyurethane material with at least one chemolysis reagent selected from the group consisting of (a) a primary or secondary organic amine, (b) an amino alcohol having a primary or secondary amino group and (c) an alcohol having at least one hydroxyl group, to form at least one organically modified carbamate.

3. The process according to claim 1, wherein the CO₂ gas stream is produced by a chemolysis of a polyurethane material, comprising at least the steps of:
providing the polyurethane material;
reacting the polyurethane material with at least one chemolysis reagent selected from the group consisting of (a) a primary or secondary organic amine, (b) an amino alcohol having a primary or secondary amino group and (c) an alcohol having at least one hydroxyl group, to form at least one organically modified carbamate;
hydrolyzing the organically modified carbamate formed beforehand to form at least organic amino compound and CO₂; and
separating off the CO₂ formed to obtain at least one CO₂ gas stream.

4. The process according to claim 3, wherein the reacting of the polyurethane material to give the organically modified carbamate and the hydrolysis of the carbamate are implemented collectively in one process step.

5. The process according to claim 2, wherein the CO₂ gas stream obtained is at a temperature of 140 °C to 220 °C.

6. The process according to claim 2, wherein reacting the polyurethane material to give the organically modified carbamate is effected using at least one catalyst.

7. The process according to claim 2, wherein the chemolysis reagent is selected from the group consisting of ethylene glycol, diethylene glycol, propylene glycol, dipropylene glycol, methyl glycol, triethylene glycol, glycerol, 2-methylpropane-1,3-diol, and mixtures of two or more of the aforementioned alcohols.

8. The process according to claim 1, wherein the CO₂ gas stream is cleaned to remove secondary constituents and the CO₂ gas stream is conducted through multiple condensers and cooled, wherein the organic and aqueous components present as a secondary constituent in a gas stream condensate and are separated off in liquid form, and, on conclusion of the cleaning, a cleaned CO₂ gas stream is sent to the reduction to form carbon monoxide.

9. The process according to claim 1, further comprising providing a hydrogen stream, wherein the hydrogen stream is converted together with the CO₂ gas stream by a reverse water-gas shift reaction to a product gas containing carbon monoxide, with or without by-products.

10. The process according to claim 9, wherein the hydrogen stream is provided by performing an electrolysis of water to give hydrogen and oxygen.

11. The process according to claim 10, wherein the water electrolysis is performed using electricity generated from renewable energy.

12. The process according to claim 1, wherein the CO₂ gas stream is introduced into an electrolysis apparatus for the reduction and reduced at an electrode to carbon monoxide.

13. An apparatus for production of phosgene, the apparatus comprising:
at least one unit for the provision of a CO₂ gas stream, which is a direct product of a process comprising at least a step of hydrolysis of at least one organic compound selected from organically modified carbamate, wherein the at least one unit for the provision of a CO₂ gas steam comprises at least one CO₂ outlet for the CO₂ gas stream;
at least one unit for reduction of the CO₂ gas stream provided to carbon monoxide, comprising at least one inlet for the CO₂ gas stream which is fluidically connected to at least one outlet of the at least one unit for the provision of a CO₂ gas stream, and at least one outlet for carbon monoxide; and
at least one unit for the production of phosgene, comprising:
at least one inlet for chlorine gas fluidically connected to a source for chlorine gas;
at least one inlet for carbon monoxide fluidically connected to at least one outlet for carbon monoxide in the unit for reduction of the CO₂ gas stream; and
at least one outlet for phosgene.

14. The apparatus according to claim 13, wherein the unit for provision of a CO₂ gas stream comprises a unit for hydrolysis of at least one organic compound, the unit for hydrolysis of at least one organic compound comprising:
at least one hydrolysis apparatus comprising at least one inlet for a water- and chemolysis reagent-containing liquid, at least one inlet for a polyurethane material and the at least one CO₂ outlet for the CO₂ gas stream, wherein the polyurethane material and the water- and chemolysis reagent-containing liquid can be contacted in a reaction zone of the hydrolysis apparatus, and the CO₂ gas stream formed can be conducted out via the at least one CO₂ outlet.

15. The apparatus according to claim 13, wherein the unit for provision of a CO₂ gas stream comprises a unit for hydrolysis of at least one organic compound, the unit for hydrolysis of at least one organic compound comprising:
at least one chemolysis apparatus comprising at least one inlet for a chemolysis reagent-containing liquid, at least one inlet for polyurethane material and the at least one outlet for organically modified carbamate, wherein the polyurethane material and the chemolysis reagent-containing liquid can be contacted in a reaction zone of the at least one chemolysis apparatus and converted to at least one organic compound selected from organically modified carbamate; and
at least one hydrolysis apparatus comprising at least one inlet for a chemolysis reagent, at least one inlet for water-containing liquid, organically modified carbamate and the at least one outlet for the CO₂ gas stream, where the organically modified carbamate and the water-containing liquid can be contacted in a reaction zone of the at least one hydrolysis apparatus, and the CO₂ gas stream formed can be conducted out via the at least one outlet for the CO₂ gas stream, wherein the inlet for organically modified carbamate is fluidically connected to the outlet for organically modified carbamate in the at least one chemolysis apparatus.

16. The apparatus according to claim 13, wherein the unit for reduction of the CO₂ gas stream is an electrolysis unit with a gas diffusion electrode for CO₂ reduction or a reverse water-gas shift reactor.

17. The process according to claim 1, further comprising cleaning the CO₂ gas stream to remove at least one secondary constituent by at least one cleaning method selected from the group consisting of condensation, adsorption, catalytic gas cleaning or gas scrubbing, to obtain a cleaned carbon dioxide, wherein the at least one secondary constituent is selected from the group consisting of alcohols, polyethers, hydrocarbons, organic amines, water, sulfur compounds, dust, oxygen and nitrogen.

18. The process according to claim 6, wherein the at least one catalyst is selected from the group consisting of carbonate, hydrogencarbonate, orthophosphate, monohydrogenorthophosphate, metaphosphate, hydroxide, organic amine, organometallic compounds or mixtures of two or more of the catalysts.

19. The process according to claim 12, wherein the electrode is a gas diffusion electrode.

20. The apparatus according to claim 13, further comprising at least one unit for cleaning of the CO₂ gas stream to remove secondary constituents, the at least one unit for cleaning of the CO₂ gas stream comprising at least one unit from the group consisting of a condensation unit, adsorption unit, gas scrubbing unit, or catalytic gas cleaning unit, where the unit for cleaning of the CO₂ gas stream has at least one inlet for the CO₂ gas stream as CO₂ gas stream to be cleaned, which is fluidically connected to the CO₂ outlet of the unit for provision of the CO₂ gas stream, and where the unit for cleaning of the CO₂ gas stream has at least one outlet for a cleaned CO₂ gas stream.
